(19) United States
(12) Patent Application Publication
(10) Pub. No.: US 2026/0048241 A1
(43) Pub. Date: Feb. 19, 2026

(54) LOW FRICTION PROFILES FOR MEDICAL DEVICES

(71) Applicant: Boston Scientific Scimed, Inc., Maple Grove, MN (US)

(72) Inventors: Scott Edward Corbeil, Litchfield, NH (US); Andrew James Schaubhut, Bolton, MA (US)

(73) Assignee: Boston Scientific Scimed, Inc., Maple Grove, MN (US)

(21) Appl. No.: 19/297,744

(22) Filed: Aug. 12, 2025

Related U.S. Application Data
(60) Provisional application No. 63/682,632, filed on Aug. 13, 2024.

(51) Int. Cl. A61M 25/09

(52) U.S. Cl. CPC A61M 25/09; A61M 2025/09083; A61M 2205/0222; A61M 2205/0238

(57) ABSTRACT

Medical devices for reducing surface contact between an outer surface of a medical device and a luminal surface of another medical device or body cavity. An illustrative medical device may comprise an elongate shaft extending from a proximal end to a distal end, a coil disposed about a distal end region of the elongate shaft, and an atraumatic tip secured to a distal end of the coil and the distal end of the elongate shaft. The elongate shaft may include a plurality of grooves and a plurality of ridges formed in an outer surface thereof and extending between the proximal end of the elongate shaft and a proximal end of the coil.

### CROSS-REFERENCE TO RELATED APPLICATIONS

This application claims the benefit of U.S. Provisional Patent Application Ser. No. 63/682,632 filed on Aug. 13, 2024, the disclosure of which is incorporated herein by reference.

### TECHNICAL FIELD

The present disclosure pertains to medical devices, and methods for manufacturing medical devices. More particularly, the present disclosure pertains to low friction profiles for medical devices

### BACKGROUND

Per- and poly-fluoroalkyl substances (PFAS) are synthetic organofluorine chemical compounds that have multiple fluorine atoms attached to an alkyl chain. PFAS may have several benefits. For example, PFAS may be, but are not limited to, water and oil repellent, non-stick, ultraviolet light stable, chemically inert, lubricious, durable, or the like. However, PFAS may have polluting effects on drinking water and/or contribute to health concerns, such as, but not limited to, high blood pressure and childhood cancers. Production of materials including PFAS has been decreased in response to the environmental and health concerns surrounding PFAS.

A wide variety of medical devices include PFAS. Some of these medical devices may require surfaces with a low coefficient of friction to function properly. There is an ongoing need to provide alternative low friction medical devices as well as alternative methods for manufacturing and using medical devices.

### BRIEF SUMMARY

This disclosure provides design, material, manufacturing method, and use alternatives for medical devices.

In a first example, a medical device may comprise an elongate shaft extending from a proximal end to a distal end, a coil disposed about a distal end region of the elongate shaft, and an atraumatic tip secured to a distal end of the coil and the distal end of the elongate shaft. The elongate shaft may include a plurality of grooves and a plurality of ridges formed in an outer surface thereof and extending between the proximal end of the elongate shaft and a proximal end of the coil.

Alternatively, or additionally to any of the examples above, in another example, at least some of the plurality of grooves and the plurality of ridges may extend parallel to a longitudinal axis of the elongate shaft.

Alternatively, or additionally to any of the examples above, in another example, at least some of the plurality of grooves and the plurality of ridges may extend circumferentially around the elongate shaft.

Alternatively, or additionally to any of the examples above, in another example, at least some of the plurality of grooves and the plurality of ridges may extend helically around and along the elongate shaft.

Alternatively, or additionally to any of the examples above, in another example, at least some of the plurality of grooves and the plurality of ridges may be longitudinally and/or circumferentially spaced by one or more regions of the elongate shaft free from the plurality of grooves and the plurality of ridges.

Alternatively, or additionally to any of the examples above, in another example, the plurality of grooves and the plurality of ridges may form a sinusoidal pattern around a circumference of the elongate shaft.

Alternatively, or additionally to any of the examples above, in another example, the plurality of grooves and the plurality of ridges may be continuously curved.

Alternatively, or additionally to any of the examples above, in another example, the plurality of grooves may each have a generally semi-circular cross-section.

Alternatively, or additionally to any of the examples above, in another example, the plurality of ridges may have a constant radius along an arc length thereof.

Alternatively, or additionally to any of the examples above, in another example, the plurality of grooves may have a varying radius along an arc length thereof.

Alternatively, or additionally to any of the examples above, in another example, the plurality of grooves may include at least one longitudinally extending groove and at least one circumferentially extending groove. The at least one circumferentially extending groove may intersect the at least one longitudinally extending groove.

Alternatively, or additionally to any of the examples above, in another example, the at least one circumferentially extending groove may have a depth less than a depth of the at least one longitudinally extending groove.

Alternatively, or additionally to any of the examples above, in another example, the elongate shaft may be a single monolithic structure.

Alternatively, or additionally to any of the examples above, in another example, the medical device may further comprise a lubricant disposed within one or more of the plurality of grooves.

Alternatively, or additionally to any of the examples above, in another example, the medical device may further comprise a laminate polymer coating disposed over the outer surface of the elongate shaft.

In another example, a medical device may comprise an elongate shaft extending from a proximal end to a distal end, a coil disposed about a distal end region of the elongate shaft, and an atraumatic tip secured to a distal end of the coil and the distal end of the elongate shaft. The elongate shaft may include a plurality of helically extending grooves and a plurality of helically extending ridges formed in an outer surface thereof and extending between the proximal end of the elongate shaft and a proximal end of the coil.

Alternatively, or additionally to any of the examples above, in another example, the plurality of helically extending grooves and the plurality of helically extending ridges may have a generally sinusoidal shape extending generally orthogonal to a longitudinal axis of at least one ridge of the plurality of helically extending ridges.

In another example, a medical device may comprise an elongate shaft extending from a proximal end to a distal end, a plurality of grooves and a plurality of ridges formed in an outer surface of the elongate shaft, a coil disposed about a distal end region of the elongate shaft, and an atraumatic tip secured to a distal end of the coil and the distal end of the elongate shaft. The plurality of grooves may include at least one longitudinally extending groove and at least one circumferentially extending groove. The at least one circumferentially extending groove may intersect the at least one longitudinally extending groove.

Alternatively, or additionally to any of the examples above, in another example, the at least one circumferentially extending groove may have a depth less than a depth of the at least one longitudinally extending groove.

Alternatively, or additionally to any of the examples above, in another example, portions of the at least one circumferentially extending groove free from intersection with the at least one longitudinally extending groove may have a constant radius.

Alternatively, or additionally to any of the examples above, in another example, the plurality of ridges may be longitudinally and circumferentially spaced around the outer surface of the elongate shaft.

Alternatively, or additionally to any of the examples above, in another example, circumferential regions of the elongate shaft free from the at least one circumferentially extending groove may be continuously curved in a sinusoidal shape about a circumference of the elongate shaft.

Alternatively, or additionally to any of the examples above, in another example, the plurality of ridges may each have a curved radially outward surface, a proximally facing generally planar lateral side, and an opposing distally facing generally planar lateral side.

The above summary of some configurations is not intended to describe each disclosed configuration or every implementation of the present disclosure. The Figures, and Detailed Description, which follow, more particularly illustrate these configurations.

### BRIEF DESCRIPTION OF THE DRAWINGS

The disclosure may be more completely understood in consideration of the following detailed description in connection with the accompanying drawings, in which:

FIG. 1 is a schematic cross-sectional view of an illustrative configuration of a medical device;

FIG. 2 is a perspective view of the illustrative configuration of the medical device of FIG. 1;

FIG. 3A is a cross-sectional view of the illustrative configuration of the medical device of FIG. 1, taken at line 3A-3A of FIG. 2;

FIG. 3B is a cross-sectional view of a medical device having an illustrative configuration of an elongate shaft that is an alternative to an illustrative configuration of the elongate shaft of the medical device depicted in FIG. 1;

FIG. 3C is a cross-sectional view of the medical device of FIG. 1 with an optional coating;

FIG. 4 is a perspective view of an illustrative configuration of a medical device;

FIG. 5 is a cross-sectional view of the illustrative configuration of the medical device of FIG. 4, taken at line 5-5 of FIG. 4;

FIG. 6 is a perspective view of an illustrative configuration of a medical device;

FIG. 7 is an enlarged view of the illustrative configuration of the medical device of FIG. 6, taken at Detail A of FIG. 6;

FIG. 8A is a cross-sectional view of the illustrative configuration of the medical device of FIG. 6, taken at line 8A-8A of FIG. 6;

FIG. 8B is a cross-sectional view of the illustrative configuration of the medical device of FIG. 6, taken at line 8B-8B of FIG. 6; and

FIG. 9 is a perspective view of an illustrative configuration of a medical device.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

Per- and poly-fluoroalkyl substances (PFAS) are synthetic organofluorine chemical compounds that have multiple fluorine atoms attached to an alkyl chain. PFAS may have several benefits. For example, PFAS may be, but are not limited to, water and oil repellent, non-stick, ultraviolet light stable, chemically inert, lubricious, durable, or the like. However, PFAS may have polluting effects on drinking water and/or contribute to health concerns, such as, but not limited to, high blood pressure and childhood cancers. Production of materials including PFAS has been decreased in response to the environmental and health concerns surrounding PFAS.

A wide variety of medical devices include PFAS. Some of these medical devices may require surfaces with a low coefficient of friction to function properly. Some illustrative medical devices that contain or use PFAS include, but are not limited to, sphincterotomes, retrieval baskets, polypectomy snares, guidewires, catheters, coated and/or jacketed extrusions, or coated and/or jacketed wires, or the like. These illustrative medical devices are not an exhaustive list, but merely representative of some types of medical devices that may include PFAS. Described herein are devices which reduce the surface area of the medical device to reduce the friction between the medical device and another medical device or a body lumen.

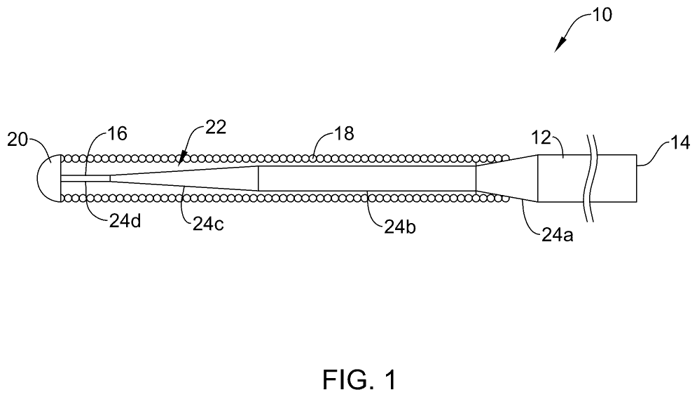

FIG. 1 is a schematic cross-sectional view of an illustrative configuration of a medical device 10. In FIG. 1, the medical device 10 may be a guidewire. Although the present disclosure includes descriptions of a medical device 10 as a guidewire, the features described herein could be implemented in other medical systems or devices where reduced friction is desired, such as, but not limited to, catheters, shafts, leads, wires, mandrels, filters, stents, or the like. The medical devices or systems may be used intravascularly and/or in any other suitable body lumen or procedure on the body including, but not limited to, pulmonary procedures, urinary procedures, endoscopic procedures, etc.

The medical device 10 may include any suitable components. For example, the medical device 10 may include a core wire or elongate shaft 12 extending from a proximal end 14 configured to remain outside the body to a distal end 16. A coil 18 may be disposed over a length of the elongate shaft 12 adjacent to a distal end region 22 of the elongate shaft 12. A tip 20 having a generally curved atraumatic shape, such as a solder tip, may be formed on and secured to the elongate shaft 12 at or adjacent to the distal end 16. A distal portion of the coil 18 may be coupled to the tip 20. In some instances, a distal portion of the coil 18 may be embedded within the tip 20. “Embedded” means to be disposed within, coupled to, set in, implanted, fixed, etc. In some examples, the tip 20 may fix the coil 18 relative to elongate shaft 12. Alternatively, or additionally, the coil 18 may be soldered, or otherwise coupled, to elongate shaft 12 proximate to the tip 20. In some instances, the coil 18 may be replaced with a slotted tube or other flexible member.

The elongate shaft 12 may be comprised of nickel-titanium alloy, stainless steel, a composite of nickel-titanium alloy and stainless steel, and/or include nickel-cobalt-chromium-molybdenum alloy (e.g., MP35-N). Alternatively, or additionally, the elongate shaft 12 may be comprised of metals, polymers, combinations or composites thereof, or other suitable materials. In some instances, a portion or all of the medical device 10 may be radiopaque to allow the medical device 10 to be viewed on a fluoroscopy screen, or other imaging technique, during a procedure. In some instances, the distal end region 22 and/or the coil 18 may be radiopaque to aid the physician in determining the location of the distal end 16 of the elongate shaft 12.

The elongate shaft 12 may be distally tapered. For example, the elongate shaft 12 may include a plurality of distal segments 24a-d or comprise a single, generally tapered distal end segment. Each distal segment may comprise a substantially constant decreased outside diameter relative to a proximal section thereof, as shown at segments 24b, 24d, or individual segments may each taper along the length of a particular segment in a distal direction, as shown at segments 24a, 24c. A vast number of alternate or additional configurations of segments and distal ends may be included without departing from the scope of the disclosure.

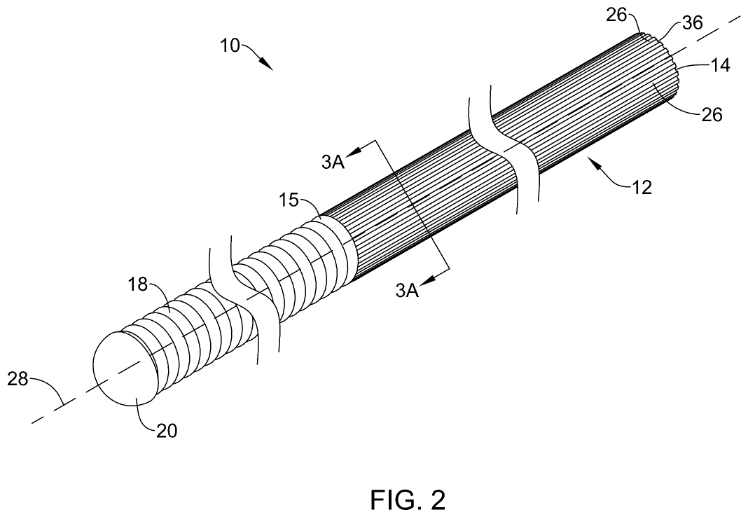

FIG. 2 is a schematic perspective view of the medical device 10 of FIG. 1. The outer surface of the elongate shaft 12 may include a plurality of ridges, splines, or flutes 26. For brevity and case of understanding not every ridge 26 is identified with a reference numeral. The plurality of ridges 26 may extend generally parallel to a longitudinal axis 28 of the medical device 10. However, this is not required. Alternatively, or additionally, the plurality of ridges may extend around the circumference of the elongate shaft 12 generally orthogonal to the longitudinal axis 28. Alternatively, or additionally, the ridges 26 may extend helically around the circumference and along a length of the elongate shaft 12. Helically extending grooves 36 and ridges 26 may provide an outer surface that resembles two or more twisted filaments. It is contemplated that the ridges 26 may extend along an entirety of the exposed elongate shaft 12 from the proximal end 14 of the elongate shaft 12 to an intermediate location 15 which abuts a proximal end of the coil 18. The intermediate location 15 may be between the proximal end 14 and the distal end 16 of the elongate shaft 12. It is contemplated that the ridges 26 may extend longitudinally along and/or circumferentially around less than an entirety of the exposed elongate shaft 12. For example, the ridges 26 may terminate or end proximal to the coil 18. It is further contemplated that the ridges 26 may terminate or end distal to the proximal end 14 of the elongate shaft 12. In yet another example, the ridges 26 may be continuous or interrupted. For example, the elongate shaft 12 may include more than one ridge at a similar circumferential (or longitudinal) location. Said differently, ridges 26 may be separated longitudinally or circumferentially by regions free from ridges 26 and/or grooves 36. Further, the ridges 26 need not be arranged in a discernible pattern.

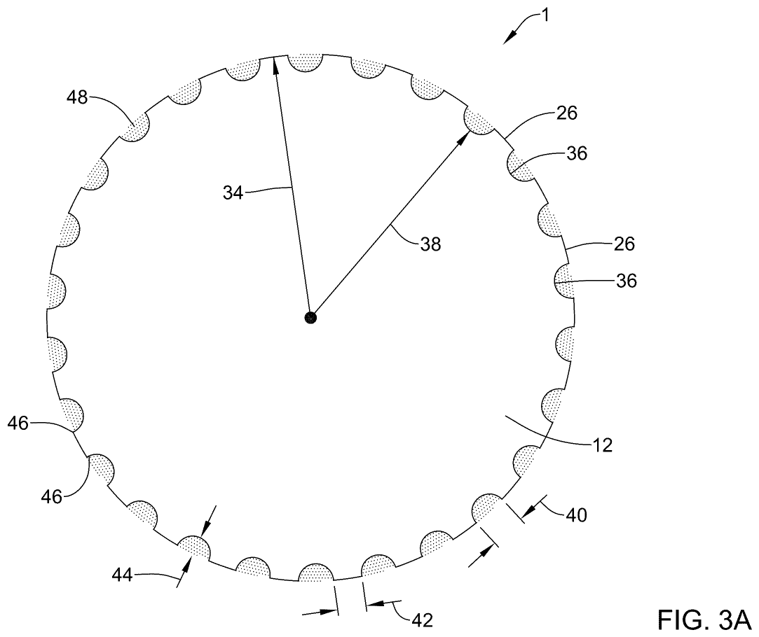

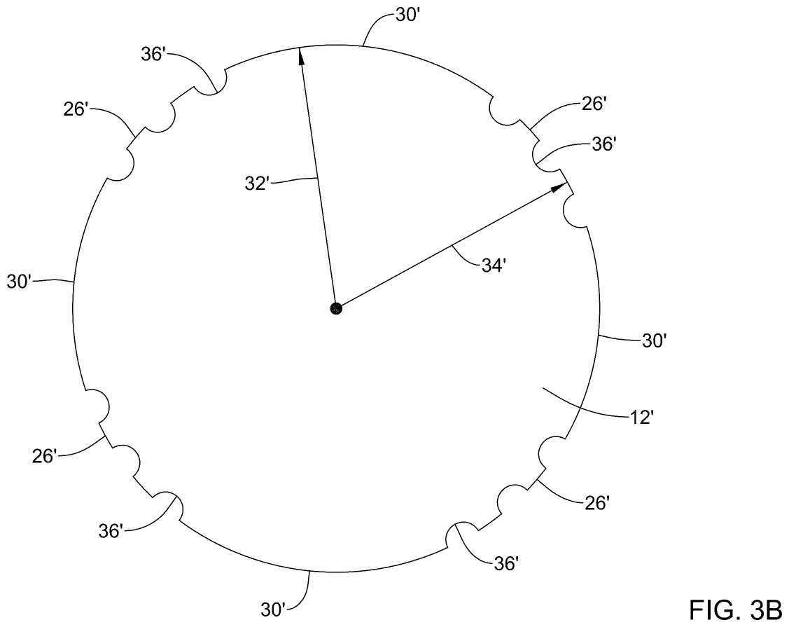

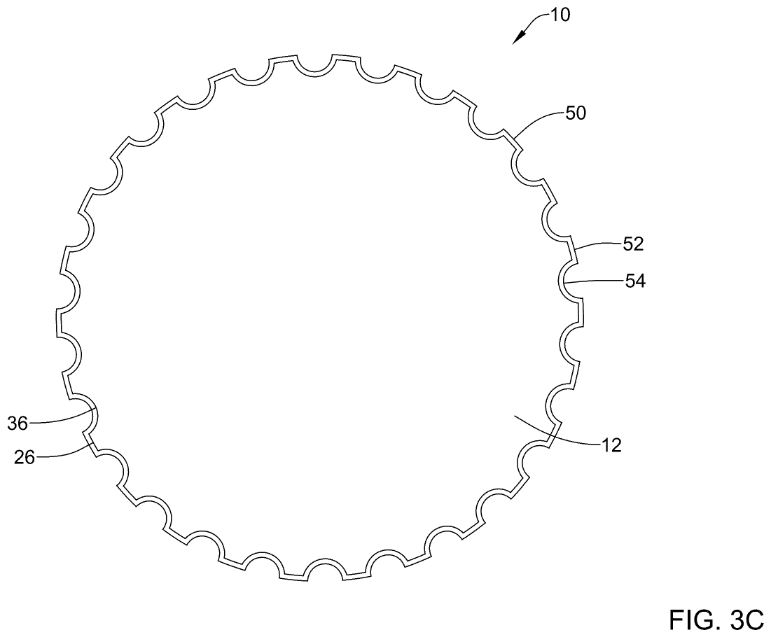

The elongate shaft 12 may include any number of ridges 26 desired. It is contemplated that the number of ridges 26 may vary depending on the arc length 42 of the ridges 26, a spacing of the ridges 26, or the like. The ridges 26 may be circumferentially spaced around a circumference of the elongate shaft 12. FIG. 3A is a schematic cross-sectional view of the elongate shaft 12 of the medical device 10, taken at line 3A-3A of FIG. 2. In some configurations, the ridges 26 may be spaced around an entirety of the circumference of the elongate shaft 12, as shown in FIG. 3A. The ridges 26 may be uniform in arc length 42 and/or spacing or non-uniform in arc length 42 and/or spacing. In other examples, the ridges 26 may be spaced around less than an entirety of the circumference of the elongate shaft 12. The ridges 26 may be spaced from one another by a plurality of reliefs, grooves, recesses, or channels 36. A vast number of alternate or additional configurations of ridges 26, grooves 36, and/or spacing thereof may be included without departing from the scope of the disclosure.

FIG. 3B illustrates a cross-sectional view of an illustrative configuration of an elongate shaft 12′ of a medical device 10′ in which groups of ridges 26′ and grooves 36′ are spaced (e.g., circumferentially spaced) from one another by regions 30′ free from ridges 26′ and grooves 36′. The regions 30′ free from ridges 26′ may have a radius 32′ that is similar to a radius 34′ of the elongate shaft 12′ adjacent to the ridges 26′. However, this is not required. In some examples, the regions 30′ free from ridges 26′ may have a radius 32′ that is less than a radius 34′ of the elongate shaft 12′ adjacent to the ridges 26′ (e.g., adjacent a peak). A vast number of alternate or additional configurations of ridges 26′, grooves 36′, and/or spacing thereof may be included without departing from the scope of the disclosure. In some configurations, the corners 46 of the ridges 26 may be rounded to reduce catch points. However, this is not required.

Returning to FIGS. 2 and 3A, the plurality of ridges 26 may be separated by a plurality of grooves 36. For brevity and ease of understanding not every groove 36 is identified with a reference numeral. The grooves 36 may be formed by removing material from the elongate shaft 12 through etching, machining, laser cutting, grinding, sandblasting or the like. In other configurations, the ridges 26 and grooves 36 may be formed using molding, thermal forming, or extrusion processes. The elongate shaft 12 including the plurality of ridges 26 and grooves 36 may be formed as single monolithic structure.

The elongate shaft 12 may have a radius 38 at the radially inward-most portion of the grooves 36 (e.g., at a trough) that is less than a radius 34 of the elongate shaft 12 at the ridges 26 (e.g., at a peak). The ridges 26 may have a substantially constant radius 34 along the arc length 42 thereof while the radius of the grooves 36 may vary along the arc length 40 thereof. The grooves 36 may have a generally semi-circular cross-sectional shape. However, this is not required. The grooves 36 may take other cross-sectional shapes as desired such as, but not limited to, square, rectangular, semi-oval, semi-oblong, triangular, polygonal, irregular, or the like. A radially outward-most portion of the grooves 36 may have an arc length 40. The arc length 40 of the radially outward-most portion of the grooves 36 may be greater than an arc length 42 of the radially outward-most portion of the ridges 26. However, this is not required. In some configurations, the arc length 40 of the radially outward-most portion of the grooves 36 may be less than the arc length 42 of the radially outward-most portion of the ridges 26. In yet other configurations, the arc length 40 of the radially outward-most portion of the grooves 36 may be approximately the same as the arc length 42 of the radially outward-most portion of the ridges 26.

The grooves 36 may have a depth 44. The depth 44 may be equal to the difference between the radius of the radius 34 of the elongate shaft 12 at the ridges 26 and the radius 38 at the radially inward-most portion of the grooves 36. When the grooves 36 have a generally semi-circular cross-sectional shape, the depth 44 may be equal to a radius of the groove 36. The depth 44 may be selected to maintain pushability of the medical device 10. For example, if the grooves 36 exceed a certain depth 44 and/or arc length 40, friction between the medical device 10 and another element may be increased and/or the grooves 36 and/or ridges 26 may create hang-up locations where the medical device 10 is caught on or mechanically engages another structure, particularly in navigating bends or curves.

The grooves 36 reduce the surface area of the elongate shaft 12 at the radially outward-most portion thereof relative to an elongate shaft 12 having a constant radius about a circumference thereof. This may reduce the contact surface area between the elongate shaft 12 and a luminal surface of another medical device or between the elongate shaft 12 and a surface of a body lumen or cavity. Reducing the contact surface area between the elongate shaft 12 (or other medical device) and another element (e.g., another medical device or a part of the anatomy) may reduce the frictional resistance between the medical device 10 and the other element. Reducing the frictional resistance between the medical device 10 and the other element may allow the medical device 10 (or other medical device) to be easily advanced relative to the other element without the use of potentially hazardous low-friction materials, such as, but not limited to, PFAS.

In some configurations, the medical device 10 may include an optional coating or lubricant 48 disposed within the grooves 36. Some illustrative biocompatible lubricants may include, but are not limited to, plant-based lecithin coatings, fatty acids, synthetic esters, polyalphaolefins (PAOs), polyalkylene glycols, silicone, or the like. Referring additionally to FIG. 3C, which illustrates a cross-sectional view of the illustrative elongate shaft 12 with an optional coating, it is further contemplated that a laminate polymer coating 50 may be disposed over the outer surface of the elongate shaft 12. The laminate polymer coating 50 may have an outer profile which conforms to the outer profile of the elongate shaft 12. For example, the laminate polymer coating 50 may include a plurality of ridges 52 and grooves 54 that are similar in form, function, and number to the plurality of ridges 26 and grooves 36 of the elongate shaft 12. However, this is not required. In some configurations, the laminate polymer coating 50 may fill the grooves 36 to provide a uniform outer diameter. The laminate polymer coating 50 may be free from fluorinated polymers. However, the laminate polymer coating 50 may provide pushability, torquability, and/or electrical isolation while reducing friction between the outer surface of the elongate shaft 12 and a luminal surface of another medical device or body lumen/cavity.

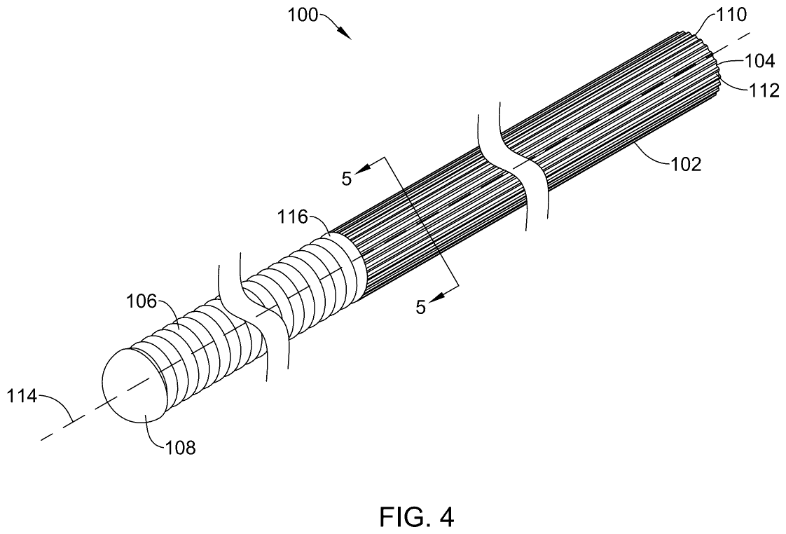

FIG. 4 is a perspective view of another illustrative medical device 100. In FIG. 4, the medical device 100 may be a guidewire. Although the present disclosure includes descriptions of a medical device 100 as a guidewire, the features described herein could be implemented in other medical systems or devices where reduced friction is desired, such as, but not limited to, catheters, shafts, leads, wires, mandrels, filters, stents, or the like. The medical devices or systems may be used intravascularly and/or in any other suitable body lumen.

The medical device 100 may include any suitable components. For example, the medical device 100 may include a core wire or elongate shaft 102 extending from a proximal end 104 configured to remain outside the body to a distal end (not explicitly shown). A coil 106 may be disposed over a length of the elongate shaft 102 adjacent to a distal end region of the elongate shaft 102. A tip 108 having an atraumatic shape (e.g., a generally curved or rounded shape, such as a solder tip or other suitable tip), may be formed on and secured to the elongate shaft 102 at or adjacent to the distal end. A distal portion of the coil 106 may be coupled to the tip 108. In some instances, a distal portion of the coil 106 may be embedded within the tip 108. The tip 108 may thus fix the coil 106 relative to elongate shaft 102. Alternatively, or additionally, the coil 106 may be soldered, or otherwise coupled, to elongate shaft 102 proximate to the tip 108. In some instances, the coil 106 may be replaced with a slotted tube or other flexible member.

The elongate shaft 102 may be comprised of nickel-titanium alloy, stainless steel, a composite of nickel-titanium alloy and stainless steel, and/or include nickel-cobalt-chromium-molybdenum alloy (e.g., MP35-N). Alternatively, or additionally, the elongate shaft 102 may be comprised of metals, polymers, combinations or composites thereof, or other suitable materials. In some instances, a portion or all of the medical device 100 may be radiopaque to allow the medical device 100 to be viewed on a fluoroscopy screen, or other imaging technique, during a procedure. In some instances, the distal end region of the elongate shaft 102 and/or coil 106 may be radiopaque to aid the physician in determining the location of the distal end of the elongate shaft 102.

While not explicitly shown, the elongate shaft 102 may be distally tapered. For example, the elongate shaft 102 may include a plurality of distal segments similar in form and function to the distal segments 24a-d described with respect to FIG. 1. A vast number of alternate or additional configurations of segments and distal ends may be included without departing from the scope of the disclosure.

The outer surface of the elongate shaft 102 may include a plurality of ridges, splines, or flutes 110. For brevity and case of understanding not every ridge 110 is identified with a reference numeral. The plurality of ridges 110 may extend generally parallel to a longitudinal axis 114 of the medical device 100. However, this is not required. In some configurations, the plurality of ridges may extend around the circumference of the elongate shaft 102 generally orthogonal to the longitudinal axis 114. In yet another example, the ridges 110 may extend helically around the circumference and along a length of the elongate shaft 102. It is contemplated that the ridges 110 may extend along an entirety of the exposed elongate shaft 102 from the proximal end 104 of the elongate shaft 102 to an intermediate location 116 which abuts a proximal end of the coil 106. The intermediate location 116 may be between the proximal end 104 and the distal end of the elongate shaft 102. It is contemplated that the ridges 110 may extend longitudinally along and/or circumferentially around less than an entirety of the exposed elongate shaft 102. For example, the ridges 110 may terminate or end proximal to the coil 106. It is further contemplated that the ridges 110 may terminate or end distal to the proximal end 104 of the elongate shaft 102. In yet another example, the ridges 110 may be continuous or interrupted. For example, the elongate shaft 102 may include more than one ridge at a similar circumferential (or longitudinal) location. Said differently, ridges 110 may be separated longitudinally or circumferentially by regions free from ridges 110 and/or grooves 112. Further, the ridges 110 need not be arranged in a discernible pattern.

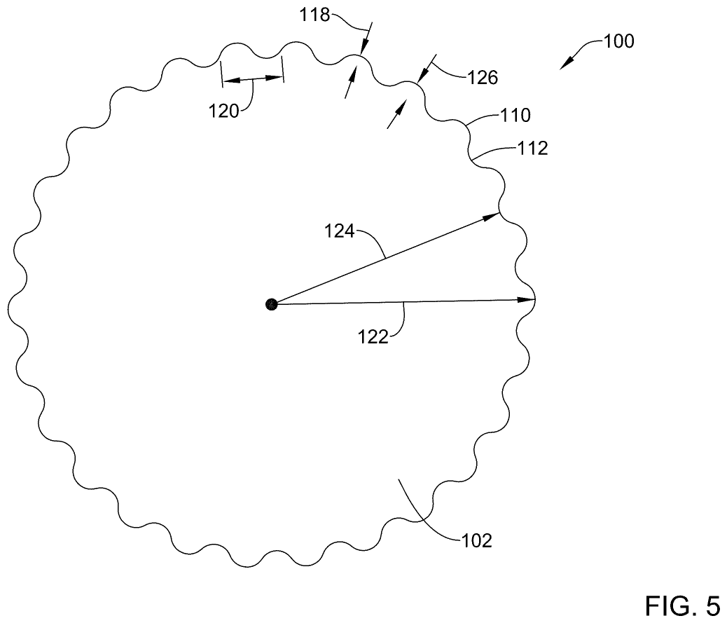

FIG. 5 is a schematic cross-sectional view of the elongate shaft 102, taken at line 5-5 of FIG. 4. Although other configurations are contemplated, the ridges 110 and grooves 112 may have an undulating or sinusoidal shape and/or the outer surface of the elongate shaft 102 may be continuously curved along the circumference thereof. In some examples, the circumference along the ridges 110 and grooves 112 may form a wave. The elongate shaft 102 may include any number of ridges 110 desired. It is contemplated that the number of ridges 110 may vary depending on the amplitude 118 of the ridges 110 or grooves 112 of the wave (e.g., an amplitude may be distance between a peak or trough and a center of the wave), the period 120 of the wave, or the like. The ridges 110 may be circumferentially spaced around a circumference of the elongate shaft 102. In some configurations, the ridges 110 may be spaced around an entirety of the circumference of the elongate shaft 102, as shown in FIG. 5. The ridges 110 may be uniform in amplitude 118, period 120, and/or spacing or non-uniform in in amplitude 118, period 120, and/or spacing. In other examples, the ridges 110 may be spaced around less than an entirety of the circumference of the elongate shaft 102. The ridges 110 may be spaced from one another by a plurality of reliefs, grooves, recesses, or channels 112. A vast number of alternate or additional configurations of ridges 110, grooves 112, and/or spacing thereof may be included without departing from the scope of the disclosure. For example, similar to that described herein with respect to FIG. 3B, groups of ridges 110 and grooves 112 may be spaced from one another by regions free from ridges 110 and grooves 112. The regions free from ridges 110 may have a radius that is similar to a radius of the elongate shaft 102 adjacent to the ridges 110. However, this is not required. In some examples, the regions free from ridges 110 may have a radius that is less than a radius of the elongate shaft 102 adjacent to the ridges 110. When the outer surface of the elongate shaft 102 has a sinusoidal wave pattern, the surface may be continuously curved such that the elongate shaft 102 is free from sharp or pointed edges which may act as catch points. However, this is not required.

The plurality of ridges 110 may be separated by a plurality of grooves 112. For brevity and ease of understanding not every groove 112 is identified with a reference numeral. The grooves 112 may be formed by removing material from the elongate shaft 102 through etching, machining, laser cutting, grinding, sandblasting or the like. In other configurations, the ridges 110 and grooves 112 may be formed using molding, thermal forming, or extrusion processes. The elongate shaft 102 including the plurality of ridges 110 and grooves 112 may be formed as single monolithic structure.

The elongate shaft 102 may have a radius 124 at the radially inward-most portion of the grooves 112 (e.g., at a trough) that is less than a radius 122 of the elongate shaft 102 at the ridges 110 (e.g., at a peak). The grooves 112 may have a continuously curved bowl-like cross-sectional shape. However, this is not required. The grooves 112 may take on other suitable cross-sectional shapes as desired such as, but not limited to, semi-circular, square, rectangular, semi-oval, semi-oblong, triangular, polygonal, irregular, or the like.

The ridges 110 and grooves 112 may have a uniform period 120, uniform amplitude 118, and/or uniform peak-to-trough distance 126. However, this is not required. In some cases, one or more of the amplitude 118, period 120, and/or peak-to-trough distance 126 may vary around the circumference of the elongate shaft 102.

The grooves 112 may have a depth or peak-to-trough distance 126. The peak-to-trough distance 126 may be equal to the difference between the radius 122 of the elongate shaft 102 at the ridges 110 and the radius 124 at the radially inward-most portion of the grooves 112. The peak-to-trough distance 126 may be selected to maintain pushability of the medical device 100. For example, if the grooves 112 exceed a certain peak-to-trough distance 126 and/or amplitude 118 and/or period 120, friction between the medical device 100 and another element may be increased and/or the grooves 112 and/or ridges 110 may create hang-up locations where the medical device 100 is caught on or mechanically engages another structure, particularly in navigating bends or curves.

The grooves 112 reduce the surface area of the elongate shaft 102 at the radially outward-most portion thereof relative to an elongate shaft 102 having a constant radius about a circumference thereof. This may reduce the contact surface area between the elongate shaft 102 and a luminal surface of another medical device or between the elongate shaft 102 and a surface of a body lumen or cavity. In some cases, the contact surface area may be a tangent contact. Reducing the contact surface area between the elongate shaft 102 (or other medical device) and another element (e.g., another medical device or a part of the anatomy) may reduce the frictional resistance between medical device 100 and the other element. Reducing the frictional resistance between the medical device 100 and the other element may allow the medical device 100 (or other medical device) to be easily advanced without the use of potentially hazardous low-friction materials, such as, but not limited to, PFAS.

In some configurations, the medical device 100 may include an optional coating or lubricant, similar in form and function to the coating or lubricant described with respect to FIG. 3A, disposed within the grooves 112. Some illustrative biocompatible lubricants may include, but are not limited to, plant-based lecithin coatings, fatty acids, synthetic esters, polyalphaolefins (PAOs), polyalkylene glycols, silicone, or the like. It is further contemplated that a laminate polymer coating similar in form and function to the coating described with respect to FIG. 3C, may be disposed over the outer surface of the elongate shaft 102. The laminate polymer coating may have an outer profile which conforms to the outer profile of the elongate shaft 102. For example, the laminate polymer coating may include a plurality of ridges and grooves that are similar in form, function, and number to the plurality of ridges 110 and grooves 112 of the elongate shaft 102. However, this is not required. In some configurations, the laminate polymer coating may fill the grooves 112 to provide a uniform outer diameter. The laminate polymer coating may be free from fluorinated polymers. However, the laminate polymer coating may provide pushability, torquability, and/or electrical isolation while reducing friction between the outer surface of the elongate shaft 102 and a luminal surface of another medical device or body lumen/cavity.

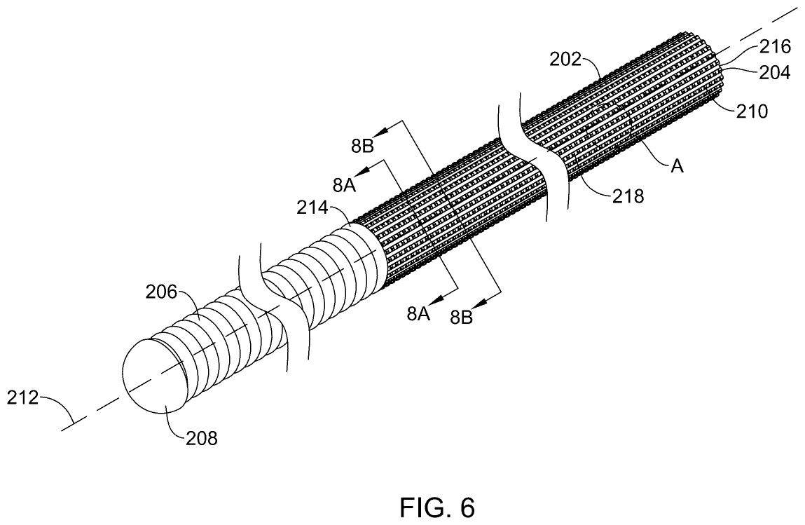

FIG. 6 is a perspective view of an illustrative configuration of a medical device 200. In FIG. 6, the medical device 200 may be a guidewire. Although the present disclosure includes descriptions of a medical device 200 as a guidewire, the features described herein could be implemented in other medical systems or devices where reduced friction is desired, such as, but not limited to, catheters, shafts, leads, wires, mandrels, filters, stents, or the like. The medical devices or systems may be used intravascularly and/or in any other suitable body lumen or procedure on the body including, but not limited to, pulmonary procedures, urinary procedures, endoscopic procedures, etc.

The medical device 200 may include any suitable components. For example, the medical device 200 may include a core wire or elongate shaft 202 extending from a proximal end 204 configured to remain outside the body to a distal end (not explicitly shown). A coil 206 may be disposed over a length of the elongate shaft 202 adjacent to a distal end region of the elongate shaft 202. A tip 208 having a generally curved atraumatic shape, such as a solder tip, may be formed on and secured to the elongate shaft 202 at or adjacent to the distal end. A distal portion of the coil 206 may be coupled to the tip 208. In some instances, a distal portion of the coil 206 may be embedded within the tip 208. The tip 208 may thus fix the coil 206 relative to the elongate shaft 202. Alternatively, or additionally, the coil 206 may be soldered, or otherwise coupled, to the elongate shaft 202 proximate to the tip 208. In some instances, the coil 206 may be replaced with a slotted tube or other flexible member.

The elongate shaft 202 may be comprised of nickel-titanium alloy, stainless steel, a composite of nickel-titanium alloy and stainless steel, and/or include nickel-cobalt-chromium-molybdenum alloy (e.g., MP35-N). Alternatively, or additionally, the elongate shaft 202 may be comprised of metals, polymers, combinations or composites thereof, or other suitable materials. In some instances, a portion or all of the medical device 200 may be radiopaque to allow the medical device 200 to be viewed on a fluoroscopy screen, or other imaging technique, during a procedure. In some instances, the distal end region and/or coil 206 may be radiopaque to aid the physician in determining the location of the distal end of the elongate shaft 202.

While not explicitly shown, the elongate shaft 202 may be distally tapered. For example, the elongate shaft 202 may include a plurality of distal segments similar in form and function to the distal segments 24a-d described with respect to FIG. 1. A vast number of alternate or additional configurations of segments and distal ends may be included without departing from the scope of the disclosure.

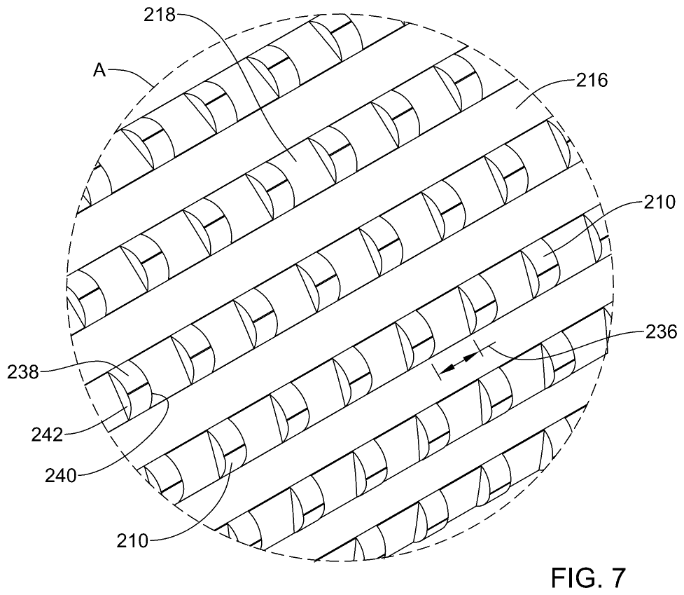

The outer surface of the elongate shaft 202 may include a plurality of longitudinally extending grooves 216 and a plurality of circumferentially extending grooves 218. The longitudinally extending grooves 216 may extend generally parallel to a longitudinal axis 212 of the medical device 200 while the circumferentially extending grooves 218 may extend around a circumference of the elongate shaft 202. The longitudinally extending grooves 216 and the circumferentially extending grooves 218 may intersect to form a plurality of ridges or protrusions 210. For brevity and case of understanding not every longitudinally extending groove 216, circumferentially extending groove 218, or ridge 210 is identified with a reference numeral. The grooves 216, 218 and ridges 210 may be more clearly shown in FIG. 7 which is a schematic enlarged view of the elongate shaft 202 taken at Detail A of FIG. 6.

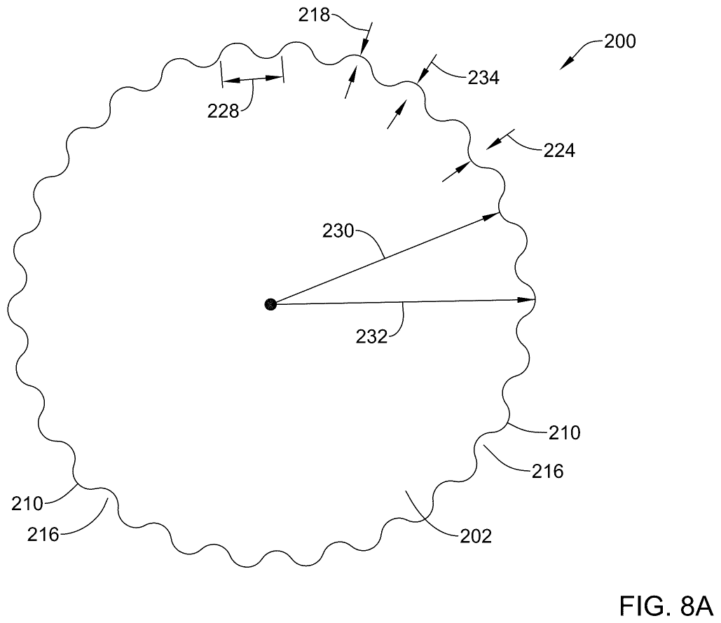

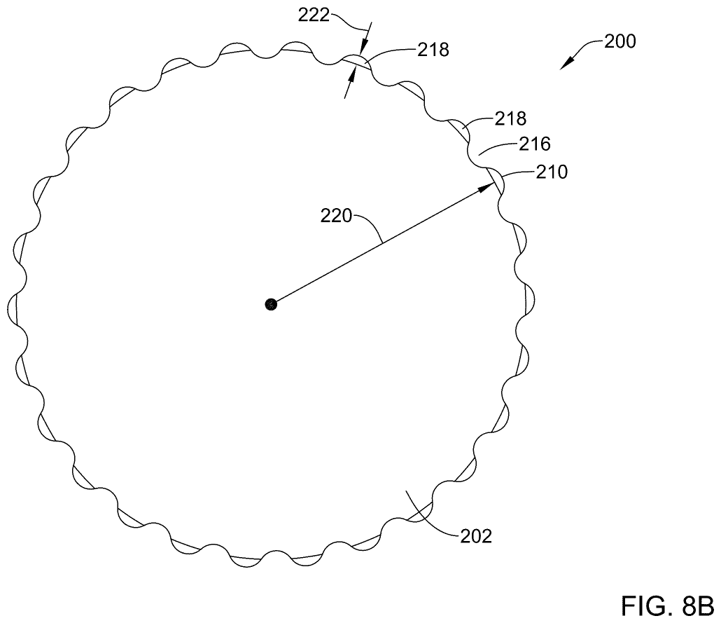

The longitudinally extending grooves 216 and the circumferentially extending grooves 218 may have different cross-sectional shapes. Referring additionally to FIG. 8A which is a cross-sectional view of the elongate shaft 202, taken at line 8A-8A of FIG. 6, the longitudinally extending grooves 216 may be formed such that collectively, the longitudinally extending grooves 216 and the ridges 210 have an undulating or sinusoidal shape. Circumferential segments of the outer surface of the elongate shaft 202 may be continuously curved along the circumference thereof. For example, circumferential regions of the elongate shaft 202 free from circumferentially extending grooves 218 may be continuously curved in a sinusoidal shape, as shown in FIG. 8A. However, this is not required. Referring additionally to FIG. 8B which is a cross-sectional view of the elongate shaft 202, taken at line 8B-8B of FIG. 6, the circumferentially extending grooves 218 may be formed such that regions where the circumferentially extending grooves 218 do not intersect with a longitudinally extending groove 216 have a generally uniform or constant radius 220 around the circumference of the elongate shaft 202. Generally, the circumferentially extending grooves 218 may have a depth 222 that is less than a depth 224 of the longitudinally extending grooves 216. However, this not required. The circumferentially extending grooves 218 may have depth 222 that is equal to or greater than the depth 224 of the longitudinally extending grooves 216.

The plurality of ridges 210 may extend generally parallel to a longitudinal axis 212 of the medical device 200 and/or around the circumference of the elongate shaft 202 generally orthogonal to the longitudinal axis 212. In another example, the ridges 210 may extend in a helical pattern around the circumference and along length of the elongate shaft 202. It is contemplated that the ridges 210 may extend along an entirety of the exposed elongate shaft 202 from the proximal end 204 of the elongate shaft 202 to an intermediate location 214 which abuts a proximal end of the coil 206. The intermediate location 214 may be between the proximal end 204 and the distal end of the elongate shaft 202. It is contemplated that the ridges 210 may extend longitudinally along and/or circumferentially around less than an entirety of the exposed elongate shaft 202. For example, the ridges 210 may terminate or end proximal to the coil 206. It is further contemplated that the ridges 210 may terminate or end distal to the proximal end 204 of the elongate shaft 202. The elongate shaft 202 may include more than one ridge at a similar circumferential (or longitudinal) location. In yet another example, the ridges 210 may be continuous or interrupted. Said differently, ridges 210 may be separated longitudinally or circumferentially by regions free from ridges 210 and/or grooves 216, 218. However, regions free from ridges 210 and/or grooves 216, 218 are not required. Further, the ridges 210 need not be arranged in a discernible pattern.

The elongate shaft 202 may include any number of ridges 210 desired. It is contemplated that the number of ridges 210 may vary depending on the amplitude 226 of the ridges 210 or grooves 216, the period 228 of a wave, the width 236 of the circumferentially extending grooves 218, or the like. The ridges 210 may be circumferentially spaced along a length and/or around a circumference of the elongate shaft 202. In some configurations, the ridges 210 may be spaced around an entirety of the circumference of the elongate shaft 202, as shown in FIGS. 6, 8A, and 8B. The ridges 210 may be uniform in amplitude 226, period 228, and/or spacing or non-uniform in in amplitude 226, period 228, and/or spacing. In other examples, the ridges 210 may be spaced around less than an entirety of the circumference of the elongate shaft 202. As described herein, the ridges 210 may be spaced from one another by a plurality of reliefs, grooves, recesses, or channels 216, 218. A vast number of alternate or additional configurations of ridges 210, grooves 216, 218, and/or spacing thereof may be included without departing from the scope of the disclosure. Similar to that described herein with respect to FIG. 3B, groups of ridges 210 and grooves 216, 218 may be longitudinally and/or circumferentially spaced from one another by regions free from ridges 210 and grooves 216, 218. The regions free from ridges 210 may have a radius that is similar to a radius 230 of the elongate shaft 202 adjacent to the ridges 210. However, this is not required. In some examples, the regions free from ridges 210 may have a radius that is less than a radius 230 of the elongate shaft 202 adjacent to the ridges 210. Where the outer surface of the elongate shaft 202 has a sinusoidal wave pattern, the surface may be continuously curved such that the elongate shaft 202 is free from sharp or pointed edges which may act as catch points. However, this is not required.

The ridges 210 may have a generally semi-cylindrical shape having a curved radially outward surface 238, a proximally facing generally planar lateral side 240, and an opposing distally facing generally planar lateral side 242. However, the ridges 210 may take other shapes as desired, such as, but not limited to, hemi-spherical, cubic, rectangular prism, pyramidal, conical, truncated pyramidal, truncated conical, polygonal shapes, polyhedrons, irregular shapes, or the like.

The grooves 216, 218 may be formed by removing material from the elongate shaft 202 through etching, machining, laser cutting, grinding, sandblasting or the like. In other configurations, the ridges 210 and grooves 216, 218 may be formed using molding, thermal forming, or extrusion processes. The elongate shaft 202 including the plurality of ridges 210 and grooves 216, 218 may be formed as single monolithic structure.

The elongate shaft 202 may have a radius 232 at the radially inward-most portion of the longitudinally extending grooves 216 (e.g., at a trough) that is less than a radius 230 of the elongate shaft 202 at the ridges 210 (e.g., at a peak). The longitudinally extending grooves 216 may have a continuously curved bowl-like cross-sectional shape. However, this is not required. The longitudinally extending grooves 216 may take other cross-sectional shapes as desired such as, but not limited to, semi-circular, square, rectangular, semi-oval, semi-oblong, triangular, polygonal, irregular, or the like. The elongate shaft 202 may have a radius 220 at the circumferentially extending grooves 218 that is greater than the radius 232 at the radially inward-most portion of the longitudinally extending grooves 216 and that is less than the radius 230 of the elongate shaft 202 at the ridges 210. However, in some cases, the radius 220 at the circumferentially extending grooves 218 may be equal to or less than the radius 232 at the radially inward-most portion of the longitudinally extending grooves 216.

The ridges 210 and longitudinally extending grooves 216 may have a uniform period 228, uniform amplitude 226, and/or uniform peak-to-trough distance 234. However, this is not required. In some cases, one or more of the amplitude 226, period 228, and/or peak-to-trough distance 234 may vary around the circumference of the elongate shaft 202. The circumferentially extending grooves 218 may have a uniform width 236 and/or a uniform depth 222. However, this is not required. In some cases, one or more of the width 236 and/or a uniform depth 222 may vary along the length of the elongate shaft 202.

The longitudinally extending grooves 216 may have a depth or peak-to-trough distance 234. The peak-to-trough distance 234 may be equal to the difference between the radius of the radius 230 of the elongate shaft 202 at the ridges 210 and the radius 232 at the radially inward-most portion of the longitudinally extending grooves 216. The peak-to-trough distance 234 and/or the depth 222 of the circumferentially extending grooves 218 may be selected to maintain pushability of the medical device 200. For example, if the grooves 216, 218 exceed a certain peak-to-trough distance 234 amplitude 226, and/or depth 222 friction between the medical device 200 and another element may be increased and/or the grooves 216, 218 and/or ridges 210 may create hang-up locations where the medical device 200 is caught on or mechanically engages another structure, particularly in navigating bends or curves.

The grooves 216, 218 reduce the surface area of the elongate shaft 202 at the radially outward-most portion thereof relative to an elongate shaft 202 having a constant radius about a circumference thereof. This may reduce the contact surface area between the elongate shaft 202 and a luminal surface of another medical device or between the elongate shaft 202 and a surface of a body lumen or cavity. In some cases, the contact surface area may be a tangent contact. Reducing the contact surface area between the elongate shaft 202 (or other medical device) and another element (e.g., another medical device or a part of the anatomy) may reduce the frictional resistance between medical device 200 and the other element. Reducing the frictional resistance between the medical device 200 and the other element may allow the medical device 200 (or other medical device) to be easily advanced without the use of potentially hazardous low-friction materials, such as, but not limited to, PFAS.

In some configurations, the medical device 200 may include an optional coating or lubricant, similar in form and function to the coating or lubricant described with respect to FIG. 3A, disposed within the grooves 216, 218. Some illustrative biocompatible lubricants may include, but are not limited to, plant-based lecithin coatings, fatty acids, synthetic esters, polyalphaolefins (PAOs), polyalkylene glycols, silicone, or the like. It is further contemplated that a laminate polymer coating similar in form and function to the coating described with respect to FIG. 3C, may be disposed over the outer surface of the elongate shaft 202. The laminate polymer coating may have an outer profile which conforms to the outer profile of the elongate shaft 202. For example, the laminate polymer coating may include a plurality of ridges and grooves that are similar in form, function, and number to the plurality of ridges 210 and grooves 216, 218 of the elongate shaft 202. However, this is not required. In some configurations, the laminate polymer coating may fill the grooves 216, 218 to provide a uniform outer diameter. The laminate polymer coating may be free from fluorinated polymers. However, the laminate polymer coating may provide pushability, torquability, and/or electrical isolation while reducing friction between the outer surface of the medical device 202 and a luminal surface of another medical device or body lumen/cavity.

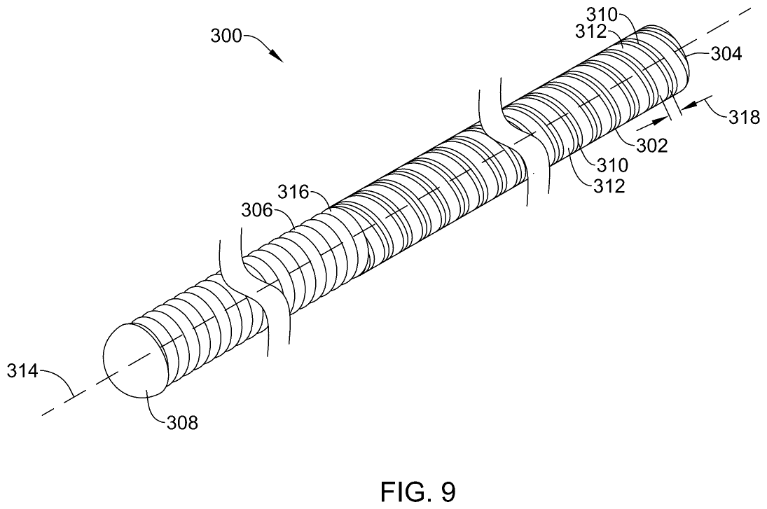

FIG. 9 is a perspective view of an illustrative configuration of a medical device 300. In FIG. 9, the medical device 300 may be a guidewire. Although the present disclosure includes descriptions of a medical device 300 as a guidewire, the features described herein could be implemented in other medical systems or devices where reduced friction is desired, such as, but not limited to, catheters, shafts, leads, wires, mandrels, filters, stents, or the like. The medical devices or systems may be used intravascularly and/or in any other suitable body lumen or procedure on the body including, but not limited to, pulmonary procedures, urinary procedures, endoscopic procedures, etc.

The medical device 300 may include a core wire or elongate shaft 302 extending from a proximal end 304 configured to remain outside the body to a distal end (not explicitly shown). A coil 306 may be disposed over a length of the elongate shaft 302 adjacent to a distal end region of the elongate shaft 302. A tip 308 having a generally curved atraumatic shape, such as a solder tip, may be formed on and secured to the elongate shaft 302 at or adjacent to the distal end. A distal portion of the coil 306 may be coupled to the tip 308. In some instances, a distal portion of the coil 306 may be embedded within the tip 308. The tip 308 may thus fix the coil 306 relative to elongate shaft 302. Alternatively, or additionally, the coil 306 may be soldered, or otherwise coupled, to elongate shaft 302 proximate to the tip 308. In some instances, the coil 306 may be replaced with a slotted tube or other flexible member.

The elongate shaft 302 may be comprised of nickel-titanium alloy, stainless steel, a composite of nickel-titanium alloy and stainless steel, and/or include nickel-cobalt-chromium-molybdenum alloy (e.g., MP35-N). Alternatively, or additionally, the elongate shaft 302 may be comprised of metals, polymers, combinations or composites thereof, or other suitable materials. In some instances, a portion or all of the medical device 300 may be radiopaque to allow the medical device 300 to be viewed on a fluoroscopy screen, or other imaging technique, during a procedure. In some instances, the distal end region and/or coil 306 may be radiopaque to aid the physician in determining the location of the distal end of the elongate shaft 302.

While not explicitly shown, the elongate shaft 302 may be distally tapered. For example, the elongate shaft 302 may include a plurality of distal segments similar in form and function to the distal segments 24a-d described with respect to FIG. 1. A vast number of alternate or additional configurations of segments and distal ends may be included without departing from the scope of the disclosure.

The outer surface of the elongate shaft 302 may include one or more ridges, splines, or flutes 310. For brevity and ease of understanding not every ridge 310 is identified with a reference numeral. In some examples, the one or more ridges 310 may be a plurality of continuous ridges 310 extending at a non-parallel and non-orthogonal angle relative to a longitudinal axis 314 of the medical device 300. In some examples, one or more continuous ridges 310 may extend helically about the outer surface of the elongate shaft 302. The ridges 310 may be circumferentially spaced about the circumference of the elongate shaft 302. However, this is not required. While the ridges 310 are illustrated as extending in a same helical direction, the elongate shaft 302 may include one or more ridges 310 extending in an opposing helical direction to create a plurality of intersecting points. In some configurations, one or more ridges 310 may extend around the circumference of the elongate shaft 302 generally orthogonal to the longitudinal axis 314. Additionally, or alternatively, one or more ridges 310 may extend longitudinally along a length of the elongate shaft 302. It is contemplated that the ridges 310 may extend along an entirety of the exposed elongate shaft 302 from the proximal end 304 of the elongate shaft 302 to an intermediate location 316 which abuts a proximal end of the coil 306. The intermediate location 316 may be between the proximal end 304 and the distal end of the elongate shaft 302. It is contemplated that the ridges 310 may extend longitudinally along and/or circumferentially around less than an entirety of the exposed elongate shaft 302. For example, the ridges 310 may terminate or end proximal to the coil 306. It is further contemplated that the ridges 310 may terminate or end distal to the proximal end 304 of the elongate shaft 302. In yet another example, the ridge(s) 310 may be continuous or interrupted. For example, the elongate shaft 302 may include more than one ridge at a similar circumferential (or longitudinal) location. Said differently, ridges 310 may be separated longitudinally and/or or circumferentially by regions free from ridges 310 and/or grooves 312. Further, the ridges 310 need not be arranged in a discernible pattern.

In some configurations, the ridges 310 and grooves 312 may have an undulating or sinusoidal shape. The sinusoidal shape may extend generally orthogonal to a longitudinal axis of the ridges 310 and/or grooves 312. The elongate shaft 302 may include any number of ridges 310 desired. It is contemplated that the number of ridges 310 may vary depending on the amplitude of the ridges 310 or grooves 312, the period of the wave, or the like. The ridges 310 may be spaced from one another by a length 318 which defines a pitch of the ridges 310. Reducing the length 318 between the adjacent ridges 310 may increase the number of the windings of the ridges 310. The ridges 310 may be uniform in amplitude, period, and/or spacing or non-uniform in in amplitude, period, and/or spacing. In other examples, the ridges 310 may be eccentrically spaced around the circumference of the elongate shaft 302. The ridges 310 may be spaced from one another by a plurality of helically extending reliefs, grooves, recesses, or channels 312. A vast number of alternate or additional configurations of ridges 310, grooves 312, and/or spacing thereof may be included without departing from the scope of the disclosure.

Similar to that described herein with respect to FIG. 3B, groups of ridges 310 and grooves 312 may be spaced from one another by regions free from ridges 310 and grooves 312. The regions free from ridges 310 may have a radius that is similar to a radius of the elongate shaft 302 adjacent to the ridges 310. However, this is not required. In some examples, the regions free from ridges 310 may have a radius that is less than a radius of the elongate shaft 302 adjacent to the ridges 310. When the outer surface of the elongate shaft 302 has a sinusoidal wave pattern, the surface may be continuously curved such that the elongate shaft 302 is free from sharp or pointed edges which may act as catch points. However, this is not required.

The grooves 312 may be formed by removing material from the elongate shaft 302 through etching, machining, laser cutting, grinding, sandblasting or the like. In other configurations, the ridges 310 and grooves 312 may be formed using molding, thermal forming, or extrusion processes. The elongate shaft 302 including the plurality of ridges 310 and grooves 312 may be formed as a single monolithic structure.

The elongate shaft 302 may have a radius at the radially inward-most portion of the grooves 312 (e.g., at a trough) that is less than a radius of the elongate shaft 302 at the ridges 310 (e.g., at a peak). The grooves 312 may have a continuously curved bowl-like cross-sectional shape. However, this is not required. The grooves 312 may take other cross-sectional shapes as desired such as, but not limited to, semi-circular, square, rectangular, semi-oval, semi-oblong, triangular, polygonal, irregular, or the like. In some configurations, the ridges 310 and grooves 312 may have shape similar to the ridges 26 and grooves 36 shown and described with respect to FIGS. 2 and 3A.

The ridges 310 and grooves 312 may have a uniform period, uniform amplitude, and/or uniform peak-to-trough distance. However, this is not required. In some cases, one or more of the amplitude, period, and/or peak-to-trough distance may vary along the length and/or around the circumference of the elongate shaft 302.

The grooves 312 may have a depth or peak-to-trough distance 126. The depth or peak-to-trough distance may be equal to the difference between the radius of the radius of the elongate shaft 302 at the ridges 310 and the radius at the radially inward-most portion of the grooves 312. The depth or peak-to-trough distance may be selected to maintain pushability of the medical device 300. For example, if the grooves 312 exceed a certain depth, peak-to-trough distance, and/or amplitude, friction between the medical device 300 and another element may be increased and/or the grooves 312 and/or ridges 310 may create hang-up locations where the medical device 300 is caught on or mechanically engages another structure, particularly in navigating bends or curves.

The grooves 312 reduce the surface area of the elongate shaft 302 at the radially outward-most portion thereof relative to the elongate shaft 302 having a constant radius about a circumference thereof. This may reduce the contact surface area between the elongate shaft 302 and a luminal surface of another medical device or between the elongate shaft 302 and a surface of a body lumen or cavity. In some cases, the contact surface area may be a tangent contact. Reducing the contact surface area between the elongate shaft 302 (or other medical device) and another element (e.g., another medical device or a part of the anatomy) may reduce the frictional resistance between medical device 300 and the other element. Reducing the frictional resistance between the medical device 300 and the other element may allow the medical device 300 (or other medical device) to be easily advanced without the use of potentially hazardous low-friction materials, such as, but not limited to, PFAS.

In some configurations, the medical device 300 may include an optional coating or lubricant, similar in form and function to the coating or lubricant described with respect to FIG. 3A, disposed within the grooves 312. Some illustrative biocompatible lubricants may include, but are not limited to, plant-based lecithin coatings, fatty acids, synthetic esters, polyalphaolefins (PAOs), polyalkylene glycols, silicone, or the like. It is further contemplated that a laminate polymer coating similar in form and function to the coating described with respect to FIG. 3C, may be disposed over the outer surface of the elongate shaft 302. The laminate polymer coating may have an outer profile which conforms to the outer profile of the elongate shaft 302. For example, the laminate polymer coating may include a plurality of ridges and grooves that are similar in form, function, and number to the plurality of ridges 310 and grooves 312 of the elongate shaft 302. However, this is not required. In some configurations, the laminate polymer coating may fill the grooves 312 to provide a uniform outer diameter. The laminate polymer coating may be free from fluorinated polymers. However, the laminate polymer coating may provide pushability, torquability, and/or electrical isolation while reducing friction between the outer surface of the elongate shaft 302 and a luminal surface of another medical device or body lumen/cavity.

The materials that can be used for the various components of medical device 10 (and/or other medical devices disclosed herein 100, 200, 300) and the various members disclosed herein may include those commonly associated with medical devices.

The various components of the devices/systems disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The disclosure's scope is, of course, defined in the language in which the appended claims are expressed.

What is claimed is:

1. A medical device, comprising:
an elongate shaft extending from a proximal end to a distal end;
a coil disposed about a distal end region of the elongate shaft; and
an atraumatic tip secured to a distal end of the coil and the distal end of the elongate shaft, and
wherein the elongate shaft includes a plurality of grooves and a plurality of ridges formed in an outer surface thereof and extending between the proximal end of the elongate shaft and a proximal end of the coil.

2. The medical device of claim 1, wherein at least some of the plurality of grooves and the plurality of ridges extend parallel to a longitudinal axis of the elongate shaft.

3. The medical device of claim 1, wherein at least some of the plurality of grooves and the plurality of ridges extend circumferentially around the elongate shaft.

4. The medical device of claim 1, wherein at least some of the plurality of grooves and the plurality of ridges are longitudinally and/or circumferentially spaced by one or more regions of the elongate shaft free from the plurality of grooves and ridges.

5. The medical device of claim 1, wherein the plurality of grooves and the plurality of ridges form a sinusoidal pattern around a circumference of the elongate shaft.

6. The medical device of claim 1, wherein the plurality of grooves and the plurality of ridges are continuously curved.

7. The medical device of claim 1, wherein the plurality of grooves each have a generally semi-circular cross-section.

8. The medical device of claim 1, wherein each ridge of the plurality of ridges has a constant radius along an arc length thereof.

9. The medical device of claim 1, wherein each groove of the plurality of grooves has a varying radius along an arc length thereof.

10. The medical device of claim 1, wherein the elongate shaft is a single monolithic structure.

11. The medical device of claim 1, further comprising a lubricant disposed within one or more of the plurality of grooves.

12. The medical device of claim 1, further comprising a laminate polymer coating disposed over the outer surface of the elongate shaft.

13. A medical device, comprising:
an elongate shaft extending from a proximal end to a distal end;
a coil disposed about a distal end region of the elongate shaft;
an atraumatic tip secured to a distal end of the coil and the distal end of the elongate shaft, and
wherein the elongate shaft includes a plurality of helically extending grooves and a plurality of helically extending ridges formed in an outer surface thereof and extending between the proximal end of the elongate shaft and a proximal end of the coil.

14. The medical device of claim 13, wherein the plurality of helically extending grooves and the plurality of helically extending ridges have a generally sinusoidal shape extending generally orthogonal to a longitudinal axis of at least one ridge of the plurality of helically extending ridges.

15. A medical device, comprising:
an elongate shaft extending from a proximal end to a distal end;
a plurality of grooves and a plurality of ridges formed in an outer surface of the elongate shaft;
a coil disposed about a distal end region of the elongate shaft; and
an atraumatic tip secured to a distal end of the coil and the distal end of the elongate shaft, and
wherein the plurality of grooves includes at least one longitudinally extending groove and at least one circumferentially extending groove, the at least one circumferentially extending groove intersecting the at least one longitudinally extending groove.

16. The medical device of claim 15, wherein the at least one circumferentially extending groove has a depth less than a depth of the at least one longitudinally extending groove.

17. The medical device of claim 15, wherein portions of the at least one circumferentially extending groove free from intersection with the at least one longitudinally extending groove has a constant radius.

18. The medical device of claim 15, wherein each ridge of the plurality of ridges is longitudinally and circumferentially spaced around the outer surface of the elongate shaft.

19. The medical device of claim 15, wherein circumferential regions of the elongate shaft free from the at least one circumferentially extending groove are continuously curved in a sinusoidal shape about a circumference of the elongate shaft.

20. The medical device of claim 15, wherein the plurality of ridges each have a curved radially outward surface, a proximally facing generally planar lateral side, and an opposing distally facing generally planar lateral side.
